(19) United States
(12) Patent Application Publication
(10) Pub. No.: US 2008/0312660 A1
(43) Pub. Date: Dec. 18, 2008

(54) DEVICES AND METHODS FOR MEASURING THE SPACE AROUND A NERVE ROOT

(75) Inventors: Jeffery L. Bleich, Palo Alto, CA (US); Gregory P. Schmitz, Los Gatos, CA (US); Eric C. Miller, Los Gatos, CA (US); Michael Villalta, San Jose, CA (US); Jefferey Bleam, Boulder Creek, CA (US); James Yurchenco, Palo Alto, CA (US); Michael P. Wallace, Pleasanton, CA (US)

Correspondence Address: SHAY GLENN LLP, 2755 CAMPUS DRIVE, SUITE 210, SAN MATEO, CA, 94403, US

(73) Assignee: Baxano, Inc., Mountain View, CA (US)

(21) Appl. No.: 12/140,201

(22) Filed: Jun. 16, 2008

Related U.S. Application Data
(60) Provisional application No. 60/944,398, filed on Jun. 15, 2007.

(51) Int. Cl. A61B 17/58; A61B 19/00; A61B 5/00

(52) U.S. Cl. 606/102; 128/898; 600/552

(57) ABSTRACT

Described herein are method, systems and devices for measuring the region adjacent to or around a nerve root, such as the space within an intervertebral foramen before, during and/or after a spinal decompression procedure. Measurement devices may be advanced by pulling on them using a guidewire passing through the intervertebral foramen and out of the subject. The measurement device may include sounds for determining one or more dimensions of the space around a nerve root within an intervertebral space, lateral recess or central canal. Various embodiments of sounds, including calibrated, inflatable, expandable, moldable, and tapered sounds (or combinations of these) are described.

### CROSS-REFERENCE TO RELATED APPLICATIONS

This application claims the benefit of U.S. Provisional Patent Application Ser. No. 60/944,398, titled “Neural Foramen Measurement Devices,” filed on Jun. 15, 2007.

### BACKGROUND OF THE INVENTION

The present invention relates generally to medical/surgical devices and methods. More specifically, the present invention relates to devices and methods for measuring the size of a compliant region adjacent to a patient's nerve root, such as the intervertebral foramina, central canal, and/or lateral recess in a spine.

In recent years, less invasive (or “minimally invasive”) surgical techniques have become increasingly more popular, as physicians, patients and medical device innovators have sought to reduce the trauma, recovery time and side effects typically associated with conventional surgery. Developing less invasive surgical methods and devices, however, poses many challenges. For example, less invasive techniques typically involve working in a smaller operating field, working with smaller devices, and trying to operate with reduced or even no direct visualization of the structures being treated. These challenges are often compounded when target tissues of a given procedure reside very close to one or more vital, non-target tissues.

One area of surgery which would likely benefit from the development of less invasive techniques is the treatment of spinal stenosis. Spinal stenosis occurs when nerve tissue and/or the blood vessels supplying nerve tissue in the spine become impinged by one or more structures pressing against them, causing symptoms. The most common form of spinal stenosis occurs in the lower (or lumbar) spine and can cause severe pain, numbness and/or loss of function in the lower back and/or one or both lower limbs.

FIG. 1 is a top view of a vertebra with the cauda equina (the bundle of nerves that extends from the base of the spinal cord) shown in cross section and two nerve roots branching from the cauda equina to exit the central spinal canal and extend through intervertebral foramina (or “neural foramina”—singular “foramen”) on either side of the vertebra. Spinal stenosis can occur when the spinal cord, cauda equina and/or nerve root(s) are impinged by one or more tissues in the spine, such as buckled or thickened ligamentum flavum, hypertrophied facet joint (shown as superior articular processes in FIG. 1), osteophytes (or “bone spurs”) on vertebrae, spondylolisthesis (sliding of one vertebra relative to an adjacent vertebra), facet joint synovial cysts, and/or collapse, bulging or herniation of an intervertebral disc. Impingement of neural and/or neurovascular tissue in the spine by one or more of these tissues may cause pain, numbness and/or loss of strength or mobility in one or both of a patient's lower limbs and/or of the patient's back.

In the United States, spinal stenosis occurs with an incidence of between 4% and 6% of adults aged 50 and older and is the most frequent reason cited for back surgery in patients aged 60 and older. Patients suffering from spinal stenosis are typically first treated with conservative approaches such as exercise therapy, analgesics, anti-inflammatory medications, and epidural steroid injections. When these conservative treatment options fail and symptoms are severe, as is frequently the case, surgery may be required to remove impinging tissue and decompress the impinged nerve tissue.

Lumbar spinal stenosis surgery involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or laminotomy). In addition, the surgery often includes partial or complete facetectomy (removal of all or part of one or more facet joints), to remove impinging ligamentum flavum or bone tissue. Spinal stenosis surgery is performed under general anesthesia, and patients are usually admitted to the hospital for five to seven days after surgery, with full recovery from surgery requiring between six weeks and three months. Many patients need extended therapy at a rehabilitation facility to regain enough mobility to live independently.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

A number of devices, systems and methods for less invasive treatment of spinal stenosis have been described by the assignee of the present invention. For example, various embodiments of such devices, systems and methods are described in U.S. patent application Ser. Nos.: 11/250,332 (Attorney Docket No. 026445-000110US), entitled “Devices and Methods for Selective Surgical Removal of Tissue,” and filed Oct. 15, 2005; 11/375,265 (Attorney Docket No. 026445-000700US), entitled “Method and Apparatus for Tissue Modification,” and filed Mar. 13, 2006; and 11/535,000 (Attorney Docket No. 026445-000900US), entitled Tissue Cutting Devices and Methods,” and filed Sep. 25, 2006, all of which applications are hereby incorporated fully be reference herein.

One challenge in treating spinal stenosis using minimally invasive tools is discerning how much space exists in the intervertebral foramen through which a given impinged nerve runs. Ideally, a surgeon performing a minimally invasive tissue removal procedure in the spine would be able to discern how impinged a given nerve is at the start of the procedure, to what extent the foramen is being cleared of tissue during the procedure, and how much room the nerve has within the foramen after the procedure is completed. At the least, a surgeon will typically want to know when the nerve is no longer being impinged by tissue and, thus, that the procedure may be complete. Making this determination in a minimally invasive setting may be quite challenging, since direct visualization of a foramen is typically not possible and soft tissues such as ligamentum flavum and nerve tissue are difficult or impossible to visualize with intraoperative fluoroscopy.

U.S. Pat. Nos. 7,166,081 and 7,172,562 describe a system of multiple rigid probes with different-sized tips for measuring an intervertebral foramen. Although such probes may work in some cases in a traditional, open surgical procedure, such rigid probes will generally not be useful for a minimally invasive or percutaneous procedure. U.S. Pat. No. 6,102,930 describes a balloon-tipped catheter device for measuring an intervertebral foramen. Again, this device is not configured to work in a minimally invasive or percutaneous procedure. As stated in the '930 patent, “A laminectomy or laminotomy is performed at the appropriate vertebral segment to allow for access to the spinal canal.” [col. 2, lines 33-35]

Therefore, it would be desirable to have devices and methods for measuring an intervertebral foramen to facilitate determination of the progress and completion of a spinal decompression procedure. Ideally, such devices and methods would work in a minimally invasive and even percutaneous access setting, without requiring large incisions, laminotomies, laminectomies, or direct visualization of the foramen. At least some of these objectives will be met by the present invention.

### SUMMARY OF THE INVENTION

Described herein are methods, devices and systems for measuring the size of a compliant region adjacent to a patient's nerve root. In particular, these devices, systems and methods may be used to measure the intervertebral foramen, and/or the lateral recess and/or the central canal of the spine. These measurements may be made to determine the size of spacing around the nerve root. The space adjacent or around the nerve root may be referred to as the compliant region. The methods, devices and systems for measuring this compliant region may be used as part of a decompression procedure in which impingement is reduced. Thus, these measurements may help gage the degree of impingement (or reduction of impingement) on the nerve root. The greater the compliant region, the less impingement. The compliant space adjacent to the nerve root may be filled with tissue (particularly soft tissues) or may be empty space. The compliant space is typically surrounded by non-compliant tissue (such as bone), forming the lateral recess, intervertebral foramina and central canal. The measurement devices and systems described herein are typically configured to be used in conjunction with a guidewire, so that they can be advanced in to the intervertebral foramen, lateral recess and/or central canal after placement of a guidewire through the intervertebral foramen. For example, the devices described herein may be configured to attach to the proximal end of a guidewire so that they can be pulled at least partially through the intervertebral foramen. The measurement device may be expandable, inflatable, calibrated to a known size and/or shape, moldable, or some combination of these. The measurement devices may include neural stimulation, which may be used to confirm the position of the device, and/or may be used to determine the dimension of the intervertebral foramen, lateral recess and/or central canal. Any of the devices described herein may form part of a system for treating a spine, or a system for measuring an intervertebral foramen. For example, a system for treating a spine may include a guidewire and any of the measurement devices described.

Also described herein are methods of measuring the size of a compliant region adjacent to a patient's nerve root. For example, the method may be used to measure the size of a patient's intervertebral foramen. These methods may also form part of an overall method of treatment of a spine. One or more of the dimensions of a subject's intervertebral space, lateral recess or central canal may be determined prior to a decompressing the spine, during the decompression of the spine, and/or after the decompression of the spine.

Described herein are methods of measuring the size of a compliant region adjacent to a patient's nerve root including the steps of: advancing a guidewire from a first position outside of the patient's body, through an intervertebral foramen, and out of the patient's body at a second position; coupling the distal end of a measurement device to the guidewire; advancing the measurement device at least partway into the intervertebral foramen, lateral recess and/or central canal, using the guidewire; and estimating a size of the region adjacent to the patient's nerve root, based on the advancement of the measurement device into the foramen. The step of advancing the measurement device may include pulling it into the intervertebral foramen, lateral recess and/or central canal behind the guidewire. In other variations, the measurement device may be advanced by sliding it over the guidewire (e.g., pushing from behind, and/or pulling distally from a second wire or connector).

In general, the guidewire may be passed through the patient by first using a cannulated probe to guide the guidewire from a first location outside of a subject's back (e.g., dorsal/posterior to the patient's intervertebral foramen), through the body, and through the intervertebral foramen. In some variations the guidewire may include a sharp (or tissue-penetrating) distal end, so that after passing through the intervertebral foramen, the guidewire may be passed through the tissue and back out of the subject from a second location dorsal/posterior to the intervertebral foramen.

Any one of the measurement devices described herein may be used as part of this method. For example, in some variations multiple measurement devices are provided, each of a different diameter, and wherein estimating the size of the foramen comprises determining a largest of the devices that can pass into the foramen.

In some variations expandable measurement devices may be used. For example, the method may include the step of expanding an expandable region of the measurement device. For example, an expandable region may be expanded by passing fluid into the expandable region of the measurement device to expand the region. The size of the measurement device (and therefore a size or dimension of the compliant region adjacent to the nerve root, e.g., the intervertebral foramen) may be estimated based on the amount of fluid that can be passed into the expandable portion.

The step of estimating the size of the compliant region adjacent to the nerve root (e.g., foramen) may include any reasonable estimation of the dimension of the region. For example, the step of estimating the size may refer to estimation of the diameter, minimum and/or maximum diameter, volume, cross-sectional area. The compliant region adjacent to the nerve root may be the intervertebral foramen, the lateral recess and/or the central canal. For example, the step of estimating the size of the compliant region adjacent to the nerve root may include estimating the size of the diameter, volume, or cross-sectional area of the intervertebral foramen adjacent or around the nerve root.

Any of the methods described herein may include the step of applying neural stimulation from the measurement device and monitoring for EMG signals. Neural stimulation may be applied from one or more discrete regions, sections, sub-regions or subsections along the measurement device. In some variations the neural stimulation is applied by use of one or more “tight bipole pairs.” Thus, current may be applied to one or more bipole pairs on the surface of the device that are only slightly separated, or separated by a small distance (e.g., less than a few millimeters, less than 1 mm, etc). The exposed surfaces of the anode and cathode forming the bipole are typically also small (e.g., less than 2 mm², less than 1 mm², etc.). In some variations, neural stimulation is applied by the measurement device to determine which portion of the measurement device a nerve within the intervertebral foramen is near-contacting or contacting; the regions may be independently activated and correlated to a known diameter. In this way, the diameter of the intervertebral foramen nearest a nerve (e.g., the nerve root) may be determined. In some variations, neural stimulation may be used to help properly advance and position the measurement device.

In some variations, the measurement device includes one or more moldable region, and the method of measuring may include the step of molding a moldable region of the measurement device within the intervertebral foramen and withdrawing the molded region. For example, the moldable region may be advanced distally (by pulling on the distal end using the guidewire), allowing the moldable region to conform to the intervertebral foramen. The moldable measuring device may be advanced distally with a light force (e.g., less than lb of force), so that the material may mold to the intervertebral foramen, and then the device may be withdrawn proximally and examined to determine a measure of the intervertebral foramen.

Any of the methods described herein may be used percutaneously. For example the guidewire and/or the measurement device may be advanced percutaneously.

Also described herein are methods of measuring the size of a compliant region adjacent to a patient's nerve root as part of a spinal decompression procedure. In some variations, this method may include the steps of advancing a guidewire from a first position outside of the patient's body, through an intervertebral foramen, and out of the patient's body at a second position, pulling the measurement device at least partially into the intervertebral foramen (wherein the measurement device is coupled to the proximal portion of the guidewire), expanding a portion of the measurement device, and estimating a size of the compliant region adjacent to the nerve root, based on the expansion of the measurement device.

Any of the methods described herein may also include the step of coupling the measuring device to the guidewire. For example, proximal end of the guidewire may be coupled to the distal end of the measuring device.

The step of expanding the portion of the measurement device may include passing a fluid into the portion. For example, fluid may be passed into an expandable balloon of the measurement device. Fluid may be passed into the portion until it reaches a predetermined pressure. In some variations, the fluid is radiopaque. Thus, the method may also include taking a radiographic image of the expanded portion using a radiographic device.

In some variations the method may also include the step of activating a transducer to estimate the size of the expanded portion. Any appropriate transducer may be used. The transducer may be included as part of the measurement device. For example, the transducer may be an optical/visual transducer (e.g., camera, CCD, etc.), a sound transducer (e.g., ultrasound, etc.), or the like. In some variations the method includes the step of rotating the transducer within an inflatable element to estimate the size of the intervertebral foramen. For example, the size may be estimated by measuring the expansion of the balloon (e.g., distance to the walls) using the intervertebral foramen.

In some variations, the step of expanding the portion of the measurement device comprises passing an expansion member into an expandable portion of the device. For example, the measurement device may include a plurality of expansion members configured as wires, rods, or the like, that may be advanced into an expandable element (e.g., bag, balloon, etc.) to expand it within the intervertebral foramen, central canal and/or lateral recess. The number of expansion members used before the device cannot be expanded any further may help provide an indication of the size of the device.

Also described herein are methods for measuring the size of a compliant region adjacent to a patient's nerve root that include electrical stimulation that may help identify the proximity of the measurement device to the nerve root as the measurement device is advanced. This electrical stimulation may prevent damaging (e.g., crushing or applying undesirable pressure) to the nerve root. For example, the method may include the steps of: advancing a guidewire from a first position outside of the patient's body, through an intervertebral foramen, and out of the patient's body at a second position, applying an electrical current between a pair of tight bipolar electrodes on a measurement device, advancing the measurement device until the patient's nerve root is stimulated by the applied electrical current, wherein the measurement device is coupled to the guidewire, and estimating a size of the region adjacent to the nerve root, based on the advancement of the measurement device.

Also described herein are measurement devices for measuring an intervertebral foramen as part of a spinal decompression procedure. In general, a measurement device may include a proximal end configured to be gripped (which may include a handle), a guidewire coupling region at the distal end (the guidewire coupling region configured to mate with the proximal end of a guidewire), and a flexible sound region near the distal end, wherein the sound region is configured to be pulled at least partially through the intervertebral foramen and provide indication of the dimension of the intervertebral foramen.

Any appropriate sound region may be used, as mentioned above. For example, the sound region of the measurement device may comprise a plurality of calibrated sounds of increasing dimension extending proximally from the distal region. In some variations, the sound region includes neural stimulation. For example, the sound region may include a plurality of bipolar pairs configured to produce a bipole filed sufficient to activate an adjacent nerve.

In some variations, the sound region may comprise an expandable region configured to be expanded (e.g., within the intervertebral foramen). The expandable region may be an inflatable balloon. In some variations, the measurement device further comprises an expansion member configured to be advanced distally and expand the expandable region. In some variations, the measurement device includes a moldable region.

Also described herein are systems for measuring the size of a compliant region adjacent to a patient's nerve root as part of a spinal decompression procedure. The system may include a guidewire having a distal end and a proximal end, and configured to pass from a first position outside of a patient's body, through an intervertebral foramen, and out of the patient's body at a second position, and a measurement device including a flexible sound region near the distal end, and a guidewire coupling region at the distal end, the guidewire coupling region configured to mate with the proximal end of the guidewire; wherein the sound region is configured to be advanced at least partially through the intervertebral foramen and provide indication of the dimension of the intervertebral foramen.

As mentioned above, any appropriate sound region may be included as part of the measurement device in the system. For example, the sound region of the measurement device may comprise a plurality of calibrated sounds of increasing dimension extending proximally from the distal region. In some variations, the sound region comprises a plurality of bipolar pairs configured to produce a bipole filed sufficient to activate an adjacent nerve. In some variations, the sound region comprises an expandable region configured to be expanded within the intervertebral foramen. In some variations the expandable region is an inflatable balloon. The measurement device may include a moldable region; in some variations the sound region is a moldable region. The measurement device may also include an expansion member configured to be advanced distally and expand the expandable region.

Any appropriate guidewire may be used. For example, the guidewire may include a shaped proximal end for coupling with the first and second flexible wires. The guidewire may also have a relatively sharp (e.g., tissue penetrating) distal end.

Also described herein are systems for measuring an intervertebral foramen as part of a spinal decompression procedure. The systems may include a guidewire having a distal end and a proximal end, and configured to pass from a first position outside of a patient's body, through an intervertebral foramen, and out of the patient's body at a second position, a first measuring device and a second measuring device. The first measuring device may include a first flexible wire having a tip coupler for coupling the wire the proximal end of the guidewire for pulling the wire into the intervertebral foramen and a first sound fixedly coupled with the first wire and having a first diameter. The second measuring device may include: a second flexible wire having a tip coupler for coupling the wire with the proximal end of the guidewire for pulling the wire into the intervertebral foramen, and a second sound fixedly coupled with the second wire and having a second diameter.

Also described herein are devices for measuring an intervertebral foramen as part of a spinal decompression procedure including: a flexible wire passable through an intervertebral foramen having a distal tip coupler for coupling with a guidewire, and a distal tapered sound region fixedly coupled with the flexible wire for passing into the intervertebral foramen, wherein the tapered sound comprises a moldable material configured to hold the shape of at least a portion of the intervertebral foramen when withdrawn from the intervertebral foramen.

Also described herein are devices for measuring an intervertebral foramen as part of a spinal decompression procedure including: a flexible catheter passable into an intervertebral foramen and having proximal and distal ends, an inflatable balloon disposed along the catheter at or near its distal end, and a coupler disposed along the catheter at or near its distal end for coupling the catheter with a guidewire. The device may also include a transducer suspended on a wire passing through the inflatable balloon for measuring the inner dimensions of the balloon. As mentioned above, the transducer may be an optical transducer (camera). In some variations, the device also includes a second balloon coupled with the catheter at or near its proximal end, wherein the second balloon inflates or deflates in response to the opposite reaction (inflation/deflation) of the inflatable balloon, when the latter is inflated in the intervertebral foramen.

Also described are devices for measuring an intervertebral foramen as part of a spinal decompression procedure, in which the devices include a flexible catheter passable through an intervertebral foramen and having proximal and distal portions, and an expandable braided portion between the proximal and distal portions. The device is configured so that pulling on the proximal and distal portions causes the expandable portion to assume an unexpanded configuration and pushing the proximal and distal portions toward one another causes the expandable portion to expand. Further, the braided portion is radio opaque.

Also described herein are devices for percutaneously measuring an intervertebral foramen as part of a spinal decompression procedure, the devices having: a flexible catheter configured to pass through an intervertebral foramen, the catheter having proximal and distal portions, and an expansion region, a plurality of long, flexible expansion members configured to pass into the expansion region, wherein the expansion region is configured to expand as the expansion members are passed therein, and a guidewire coupling region configured to couple the catheter with a guidewire that can advance the catheter into the foramen.

In some variations, the guidewire coupling region comprises a guidewire coupler at or near the distal end of the catheter for allowing the catheter to be pulled into the foramen behind the guidewire. In other variations, the guidewire coupling region comprises a guidewire lumen for allowing the catheter to be passed into the foramen over a guidewire.

Any of the methods, systems and devices described above for use in the intervertebral foramen may also be used (and/or adapted for use) to determine the size of a compliant region adjacent to a nerve root within other regions other than just the intervertebral foramen. For example, these systems, devices and methods may be used to determine the size or dimensions of the lateral recess or central canal (particularly the portion of these structures near the nerve root).

### INCORPORATION BY REFERENCE

All publications and patent applications mentioned in this specification are herein incorporated by reference in their entirety, as if each individual publication or patent application was specifically and individually indicated to be incorporated by reference.

### BRIEF DESCRIPTION OF THE DRAWINGS

FIG. 1 is a cross-sectional view of a spine, showing a top view of a lumbar vertebra, a cross-sectional view of the cauda equina, and two exiting nerve roots.

FIG. 2 is a side view of a portion of a lumbar spine without nerve root impingement, showing two adjacent vertebrae, an intervertebral disk, and a nerve root exiting an intervertebral foramen.

FIG. 3 is a side view of a portion of a lumbar spine as in FIG. 2, but demonstrating impingement of the nerve root by various tissues as in a case of spinal stenosis.

FIG. 4 is a cross-sectional view of a portion of a spine and back, with a tissue removal device in position for removing ligamentum flavum and/or bone tissue to treat spinal stenosis and/or neural/neurovascular impingement.

FIG. 5 is a side view of a portion of a lumbar spine as in FIG. 2, with a device for measuring a foramen shown in cross-section.

FIG. 6 is a side view of a lumbar spine and device as in FIG. 5, but demonstrating impingement of the nerve root by various tissues as in a case of spinal stenosis.

FIG. 7A is a perspective view of a device for measuring the compliant region adjacent to a nerve root (e.g., in an intervertebral foramen), according to one embodiment of the present invention.

FIG. 7B is a cross-sectional view of a spine, showing the device of FIG. 7A in place for measuring space in a foramen.

FIG. 8 is a side view of a system for measuring the compliant region adjacent to a nerve root including multiple sound devices, according to one embodiment.

FIG. 9 is a side view of a device for measuring the compliant region adjacent to a nerve root (e.g., a foramen) including multiple slideable sounds, according to one embodiment.

FIG. 10 is a side view of a tapered, dilation device for measuring an intervertebral foramen, according to one embodiment.

FIG. 11 is a side view of a tapered, expanding device for measuring an intervertebral foramen.

FIG. 12A is a cross-sectional view of a spine with an intervertebral measurement device.

FIG. 12B is a side view of a portion of a spine, showing an inflatable balloon portion of the device of FIG. 12A in cross section within an intervertebral foramen.

FIG. 13 is a side view of a proximal/distal balloon-type device for measuring an intervertebral foramen.

FIG. 14A is a side view of a balloon-type device for measuring an intervertebral foramen including internal electrodes.

FIG. 14B is another variation of a balloon-type device for measuring intervertebral foramen,

FIG. 14C is a side view of another variation of a balloon-type device with a built-in miniature camera for measuring an intervertebral foramen.

FIGS. 15A and 15B are side views of a measurement device having an expandable mesh portion.

FIG. 16 is a side view of a measurement device having an expandable pouch and multiple elongate expansion members.

FIG. 17 is a perspective view of a distal portion of a tissue removal device having an expandable portion for helping measure the compliant region adjacent to a nerve root.

FIG. 18 is a perspective view of a distal portion of a tissue removal device having an expandable portion for helping measure the compliant region adjacent to a nerve root.

FIG. 19 is a perspective view of a distal portion of a tissue removal device having an expandable portion for helping measure the compliant region adjacent to a nerve root.

FIG. 20A is another variation of a device for measuring the compliant region adjacent to a nerve root (e.g., in an intervertebral foramen) including a plurality of tight bipole pairs.

FIG. 20B and 20C show enlarged views of the top and bottom (respectively) of the distal end of the device of FIG. 20A.

FIG. 21 illustrates the component parts of one exemplary system for measuring.

FIG. 22 illustrates operation of one variation of a device for measuring.

FIGS. 23A and 23B further illustrate the method of operation shown in FIG. 22.

FIGS. 24A to 24C illustrate another variation of a measurement device.

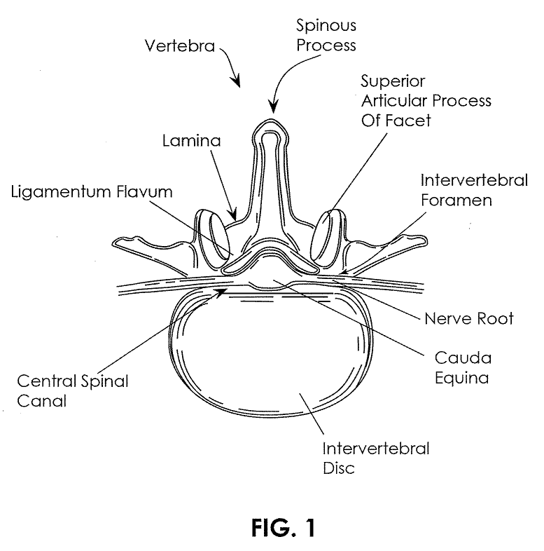

### DETAILED DESCRIPTION OF THE INVENTION

The present invention is directed primarily to medical/surgical devices, systems and methods for measuring the compliant region adjacent to a nerve root before, during and/or after a spine tissue removal procedure (or “decompression procedure”) of a constricted region surrounding the nerve root (e.g., within an intervertebral foramina, spinal canal and/or lateral recess). The devices, methods and systems described herein may be used with any appropriate spinal treatment, including those described in: U.S. patent application Ser. No.: 11/251,205, entitled “Devices and Methods for Tissue Access,” and filed Oct. 15, 2005; U.S. patent application Ser. No.: 11/457,416, entitled “Spinal Access and Neural Localization,” and filed Jul. 13, 2006; U.S. patent application Ser. No.: 11/468,247, entitled “Tissue Access Guidewire System and Method,” and filed Aug. 29, 2006; U.S. patent application Ser. No.: 11/251,165, entitled “Devices and Methods for Tissue Modification,” and filed Oct. 15, 2005; U.S. patent application Ser. No.: 11/375,265, entitled “Methods and Apparatus for Tissue Modification,” and filed Mar. 13, 2006; U.S. patent application Ser. No.: 11/535,000, entitled “Tissue Cutting Devices and Methods,” and filed Sep. 5, 2006; and U.S. patent application Ser. No.: 11/687,558, entitled “Flexible Tissue Removal Devices and Methods,” and filed Mar. 16, 2007, all of which applications are hereby incorporated by reference herein in their entirety.

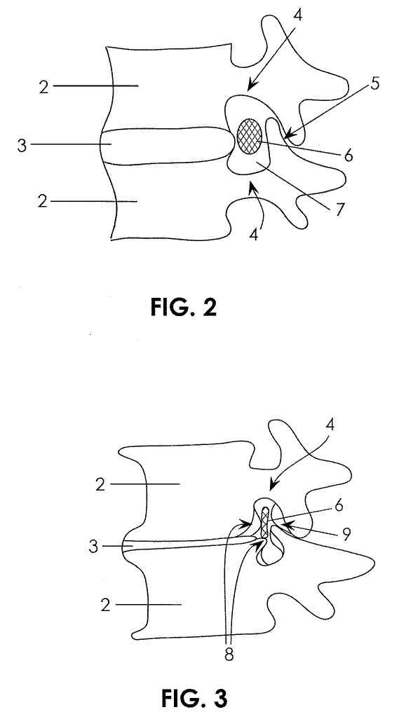

FIG. 2 is a side view of a portion of a lumbar spine without nerve root impingement, showing two adjacent vertebrae, an intervertebral disk, and a nerve root exiting an intervertebral foramen. Visible in this view are vertebral bodies 2, pedicles 4, a facet joint 5, and a nerve root 6 passing through an open intervertebral foramen 7.

FIG. 3 is a side view of the same portion of lumbar spine with nerve impingement as in a case of lateral recess and foraminal spinal stenosis. In this figure, there is collapse of disc space and bone osteophytes 8 with facet hypertrophy (enlargement) causing severe compression of nerve root 6. Ligamentum flavum 9 may also buckle, collapse and/or hypertrophy, thus further impinging on nerve root 6.

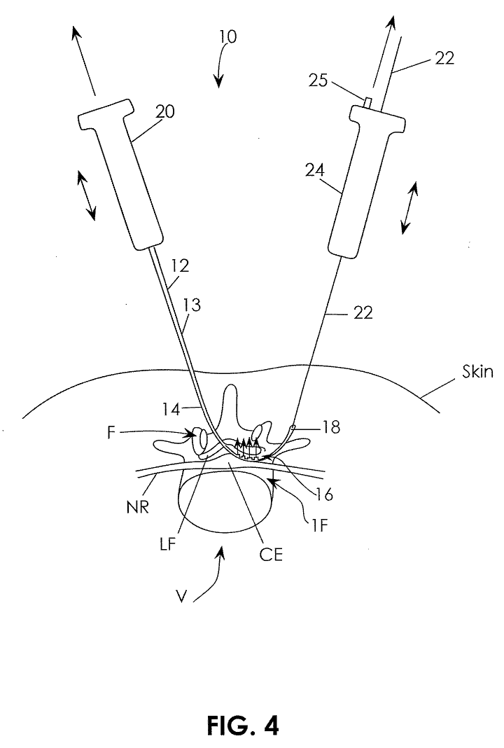

Referring to FIG. 4, one embodiment of a tissue removal device 10 for performing a minimally invasive or percutaneous spinal decompression procedure is shown. Device 10 may suitably include a proximal handle 20 coupled with a shaft 12 having a proximal, rigid portion 13 and a distal, flexible portion 14 on which one or more tissue modifying members 16 may be disposed. A guidewire coupler 18 may be formed in (or attached to) flexible portion 14 at or near its distal end, for coupling with a guidewire 22, which in turn may be coupled with a guidewire handle 24 (or “distal handle”), which may include a tightening lever 25 for tightening handle 24 around guidewire 22.

Device 10 is shown percutaneously placed in position for performing a tissue modification procedure in a patient's spine, with various anatomical structures shown including a vertebra V, cauda equina CE, ligamentum flavum LF, nerve root NR, facet F, and intervertebral foramen IF. Various embodiments of device 10 may be used in the spine to remove ligamentum flavum LF, facet bone F, bony growths, or some combination thereof, to help decompress cauda equina CE and/or nerve root NR tissue and thus help treat spinal stenosis and/or neural or neurovascular impingement. Although this use of device 10 will not be continuously repeated for every embodiment below, any of the described embodiments may be used to remove ligamentum flavum alone, bone alone, or a combination of ligament and bone in the spine to treat neural impingement, neurovascular impingement and/or spinal stenosis.

In one embodiment of a method for modifying tissue using device 10, a distal end of 22 guidewire may be placed into the patient, along a curved path between target and non-target tissue, and out of the patient. A distal portion of guidewire 22 may then be coupled with guidewire handle 24, such as by passing guidewire 22 through a central bore in handle 24 and tightening handle 24 around guidewire 22 via tightening lever 25 or other tightening means. A proximal end of guidewire 22 may then be coupled with coupling member 18 and used to pull distal shaft portion 14 between target and non-target tissues. In some embodiments, device 10 may be advanced into the patient percutaneously, while in alternative embodiments, device 10 may be advanced through a small incision or larger incision. Once advanced into the patient, flexible distal shaft portion 14 may be advanced along a curved path between the target and non-target tissues, and in some instances may be pulled at least partway into an intervertebral foramen IF of the spine.

Proximal handle 20 and guidewire handle 24 may be pulled (or “tensioned”—solid/single-tipped arrows) to urge tissue modifying members 16 against the target tissue (in this case, ligamentum flavum LF). Generally, tissue modifying members 16 may be fixedly attached to (or formed in) one side or surface of distal portion 14, while an opposite side or portion of distal portion 14 faces non-target tissue, such as cauda equina CE and/or nerve root NR. The opposite side of distal portion 14 will generally be atraumatic and/or include an atraumatic cover, coating, shield, barrier, tissue capture member or the like. With tensioning force applied to device 10, handles 20, 24 may be used to reciprocate device 10 back and forth (solid/double-tipped arrows) to cause tissue modifying members 16 to cut, remove, shred or otherwise modify the target tissue. In various embodiments, for example, target tissue may include only ligamentum flavum LF, only bone, or a combination of both.

Reciprocation and tensioning may be continued until a desired amount of tissue is removed. Removed target tissue, in some embodiments, may be collected, captured or trapped between tissue modifying members 16 and/or in one or more tissue capture members or chambers (not shown). When a desired amount of target tissue has been removed, which may be determined, for example, by tactile feedback provided to the surgeon by device 10, by radiographic imaging, and/or by direct visualization (such as in an open surgical case), guidewire 22 may be released from distal handle 24, and device 10 may be removed from the patient's back. If desired, device 10 may be passed into the patient's spine again for additional tissue modification, and/or other devices may be passed into the spine.

In general, all of the devices, systems and methods described herein may be adapted for use with a guidewire and/or bimanual operation similar to that described above. The intervertebral foramina region is extremely narrow, and includes one or more nerves, such as the nerve root. When maneuvering within the intervertebral foramen, it is extremely important to avoid damage to the nerve root. The use of a guidewire and/or bimanual manipulation approach is one way to prevent damage to the nerve root. A bimanual approach allows both proximal and distal manipulation of the device (e.g., measuring device) from outside of the patient. The bimanual manipulation may be performed using a guidewire by coupling the distal end of a device to the proximal end of the guidewire, and tensioning the guidewire distally. Bimanual manipulation may also allow the device to navigate the foramen, which may be irregularly shaped and curved. Measuring devices that are not sufficiently flexible (and particularly devices having rigid or stiff distal regions) may not provide accurate measurements.

Any of the devices and systems described herein may be adapted for bimanual manipulation. For example, the distal region of any of the measurement devices described herein may be flexible or bendable. Sounds or sounding regions on these devices may be rigid or incompressible (to provide accurate estimates of foramen size), however the sound may be located on a flexible string, backbone, cannula, etc. In some variations the proximal region is less flexible (and may even be rigid) than the distal region. The proximal region may also include a handle, as described in greater detail below. In some variations, the distal end (or a region near the distal end) includes a coupling region that is configured to couplet to a guidewire, and particularly the proximal end of a guidewire. Exemplary couplers may also be found, for example, in U.S. patent application Ser. No. 12/127,535, filed May 27, 2008, and titled “GUIDEWIRE EXCHANGE SYSTEMS TO TREAT SPINAL STENOSIS”. In general, these couplers may include a mating region for mating with a portion of the guidewire. For example, the mating region may be a channel or opening into which the proximal end of the guidewire may be seated. The channel may include a lock or locking member configured to secure the guidewire to the coupler. In one variation the coupler is a seat that includes channel with a proximal opening. The window narrows distally. A guidewire may include an enlarged proximal end (e.g., a ball or cylinder of larger diameter attached to the proximal end) that can seat into the coupler by passing through the proximal window and sliding distally until it is secured in the narrowing channel by friction between the walls of the channel and the proximal end of the guidewire.

Any of the devices described herein may also be adapted to stimulate a nerve root. Stimulation may be provided to orient or guide the measurement device (e.g., to prevent damage to the nerve as the device is positioned). In some variations, the stimulation may be provided and controlled to determine the size of the foramen relative to the measurement device. This is described in greater detail below.

Any of the devices described herein may also be used with a visualization technique such as fluoroscopy. For example, a fluoroscope may be used to visualize the intervertebral foramen to help guide the measuring device, or to provide visual output on the size. Thus, the measurement devices described herein may be adapted to allow direct visualization. For example, the devices may include indicator regions that can be visualized (e.g., under fluoroscopy) or calibration regions having a known measurement providing calibration of the fluoroscopic image. Other variations are described below.

Any of the devices described herein may also include a moldable or formable region which may be inserted into the intervertebral foramen region (or lateral recess, or central canal) in order to make a partial or complete mold of the space which can be withdrawn and examined. For example, a distal portion of the measurement device maybe moldable (e.g., made of a pliable or formable material).

Described below are variations of measuring devices for measuring the compliant region adjacent to a nerve root, when the nerve root is surrounded by bone or other hard tissue that may impinge on the nerve root, such as within the intervertebral foramen. Variations of measuring devices may be inflatable, expandable, calibrated to a known shape/size, moldable/formable, or any combination of these. As mentioned, any of these variations may be adapted for bimanual use, and may include neurostimluation to determine position and/or to determine the size of the region adjacent to the nerve.

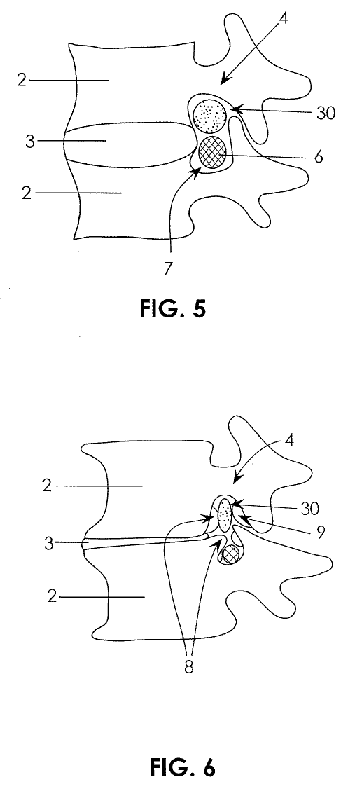

With reference now to FIGS. 5 and 6, two portions of a lumbar spine are shown, similar to those shown in FIGS. 2 and 3. As mentioned above, it may be desirable before, during or after a spine tissue removal procedure, such as a procedure performed with device 10 of FIG. 4 or with any other suitable device, to measure one or more intervertebral foramina to help determine how complete the procedure is and/or how much additional tissue might ideally be removed. In FIGS. 5 and 6, an expandable foramen measurement device 30 is shown in cross section within an intervertebral foramen 7. In FIG. 5, where there is no nerve root impingement and plenty of room in foramen 7, device 30 can expand to a larger size, compared to its expansion in FIG. 6, where bone and ligamentum flavum tissue has grown into foramen 7 and impinged on nerve root 6. By measuring an amount of fluid passable into device 30 and/or by imaging the expandable portion of device 30 using radiographic methods, one may measure an intervertebral foramen 7 before, during and/or after a spinal decompression procedure to gauge how complete the procedure is and/or how much additional tissue would ideally be removed.

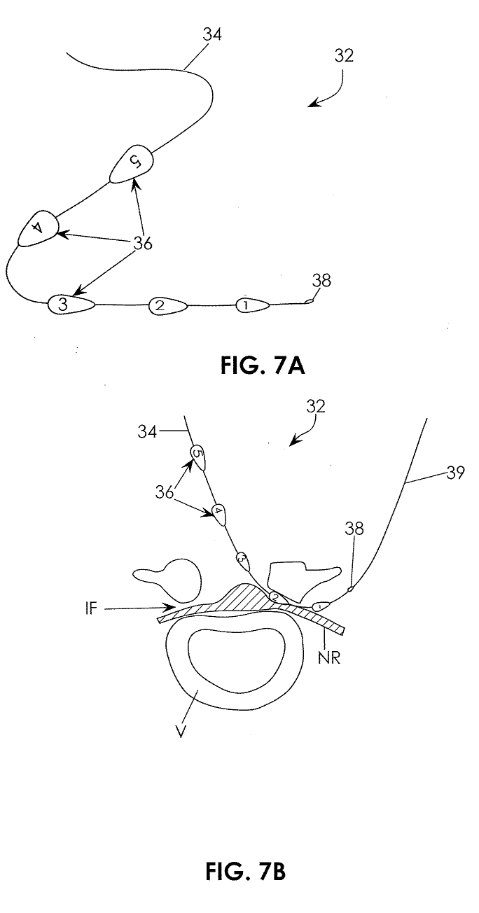

FIGS. 7A and 7B illustrate one variation of a device 32 for measuring an intervertebral foramen (IF). This variation includes calibrated (preformed to a known shape/size) sounds, and is shown in perspective view in FIG. 7A, and illustrated in position in a spine in FIG. 7B. In one embodiment, device 32 includes a flexible wire 34 at (at least) the distal end of the device, multiple sounds 36 (or “sound members”) fixedly coupled with wire 34, and a guidewire coupler 38. The sound members may be preformed to a known (calibrated) diameter, and/or shape. Various embodiments of guidewire coupler 38, and methods for using them to couple a device with a guidewire, are described in greater detail, for example, in U.S. patent application Ser. No. 11/468,247, which was previously incorporated by reference. In FIG. 7B, device 32 is shown in a spine, coupled with a guidewire 39. Guidewire 39 may be used to pull device 32 into a spine percutaneously or through a minimally invasive incision, thus obviating the need for the large incision, laminectomy and/or laminotomy required for using prior art devices.

In various embodiments, device 32 may include any number of sounds 36, each having any suitable shape and diameter. In the embodiment shown, for example, sounds 36 have a slightly tapered, bullet-like shape and are labeled with numbers 1-5. In some embodiments, such number labels may be radiopaque so as to be easily visible via intraoperative fluoroscopy. In other embodiments, sounds 36 may be completely radiopaque. Sounds 36 may have a tapered shape to facilitate their passage into an intervertebral foramen (IF) and between nerve root (NR) and impinging tissue. In other embodiments, sounds 36 may be cylindrical, ovoid, spherical, square, rectangular or any of a number of shapes. In some embodiments, sounds 36 may increase in size along flexible wire 34. For example, in one embodiment, sounds 36 may have diameters of approximately 1 mm, 2 mm, 3 mm, 4 mm and 5 mm. In various embodiments, any number of sounds 36 may be coupled with flexible wire 34, such as but not limited to between two and twenty sounds 36. The size of an intervertebral foramen may be assessed or approximated by determining the largest sound 36 that can pass into the foramen. This may be determined, in various embodiments, by tactile feel, radiographic imaging, depth markers on flexible wire 34 and/or the like. In various embodiments, sounds 36 and wire 34 may be made of any suitable material, such as but not limited to metals, such as stainless steel and Nitinol, or polymers. In some embodiments, sounds 36 may be completely rigid, such as those made of stainless steel, while in alternative embodiments sounds 36 may have some amount of “give” or flexibility, for example sounds made of a compliant polymer or filled with a gel or fluid.

In an alternative embodiment, device 32 may be passed into the spine over a guidewire and may, thus, include a guidewire lumen. Any of the devices or systems described herein may be adapted so that they can be either passed over a guidewire. In some variations the devices are adapted to be pulled into a spine behind a guidewire, as mentioned before.

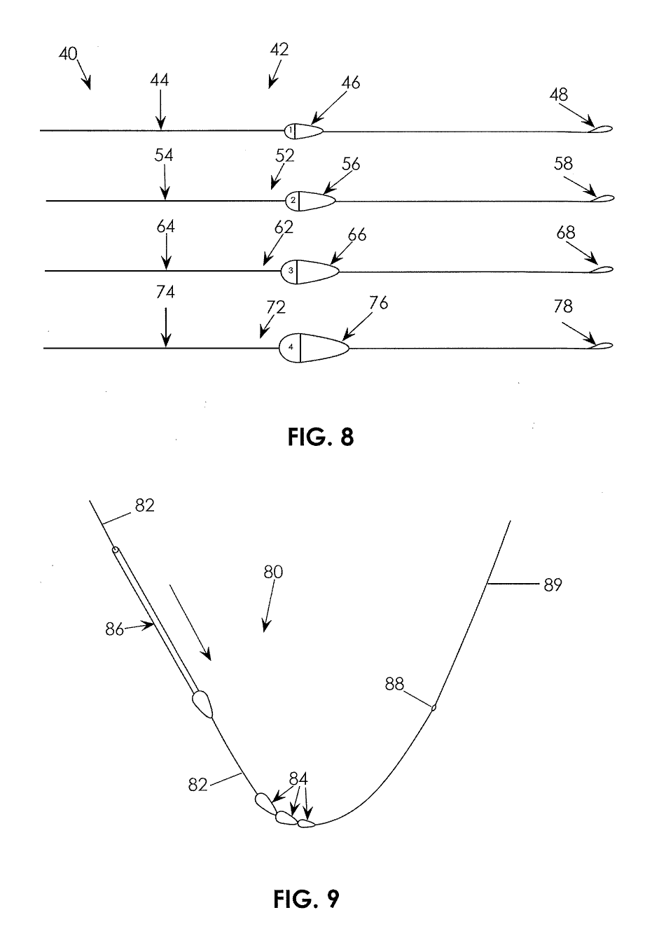

FIG. 8 is an alternative embodiment, including a system 40 for measuring a foramen, and includes multiple sound devices 42, 52, 62, 72. Each sound device 42, 52, 62, 72 may include a flexible wire 44, 54, 64, 74, a sound 46, 56, 66, 76 fixedly coupled with the wire, and a guidewire coupler 48, 58, 68, 78 disposed at or near a distal tip of the wire. As with the previously described embodiment, sounds 46, 56, 66, 76 may have any size and shape. In one embodiment, system 40 may include multiple devices 42, 52, 62, 72 with gradually increasing sizes of sounds 46, 56, 66, 76, so that each device may be passed sequentially into a spine to determine the largest sound that may pass into an intervertebral foramen. In various embodiments, any number of devices 42, 52, 62, 72 having any sizes of sounds 46, 56, 66, 76 may be provided, such as but not limited 1 mm, 2 mm, 3 mm, 4 mm, sounds, etc. In this embodiment, each sound device 42, 52, 62, 72 is inserted and then removed before the next largest device is inserted.

With reference to FIG. 9, in another variations, a foramen measurement device 80 includes a flexible wire 82 (at the distal end), multiple sounds 84 slideably disposed over wire 82, a pusher 86 slideably disposed over wire 82, and a guidewire coupler 88 for attaching device 80 to a guidewire 89. In this embodiment, sounds 84 of increasing diameter may be advanced into a spine and into a foramen using pusher 86, and sounds 84 may be used to determine an approximate size of the foramen as discussed above. In this embodiment, device 80 may remain in place in the spine while sounds 84 are advanced sequentially along it into the foramen.

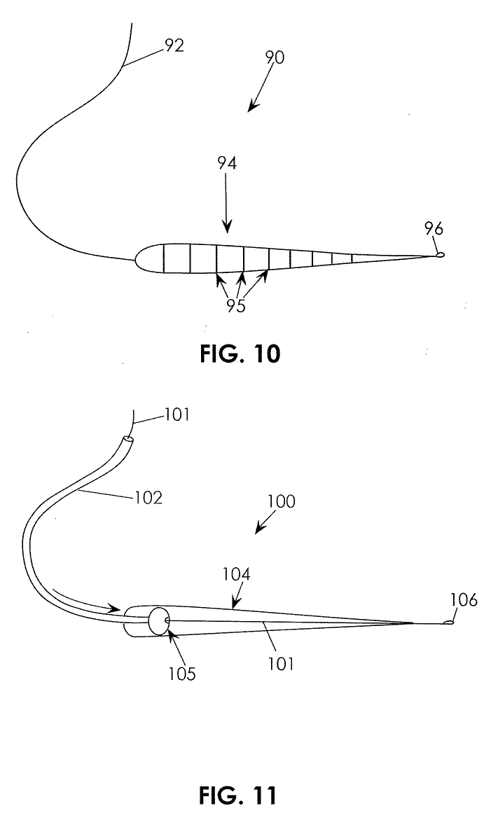

In some variations, the measurement device includes a tapered or tapering region that is calibrated to determine the minimum diameter of the intervertebral foramen. For example, FIG. 10 shows another alternative embodiment of an intervertebral measurement device 90 that includes a flexible wire 92, a long, tapered sound member 94 fixedly coupled with wire 92, and a guidewire coupler 96 distal tip 96. The tapering sound member may be flexible (e.g., along the length). The sound member 94 may include multiple radiopaque markers 95, so that sound 94 may be passed into an intervertebral foramen until it cannot pass any further, and a radiographic image may then be taken (such as by fluoroscopy) to determine an approximate size of the foramen. In this or another embodiment, depth markers may also be placed on wire 92 to help determine how far sound 94 is able to pass into a foramen. In some cases, device 90 may be used not only to measure an approximate size of a foramen but may also be used to dilate a space within the foramen, thus making it easier to pass subsequent instruments, such as a tissue removal device.

FIG. 11 is another embodiment of a measuring device 100 which includes a flexible wire 101, an expandable portion 104, an expander 105 slideably disposed over wire 101 and within expandable portion 104, a pusher 102 for advancing expander 105 along wire 101, and a guidewire coupler 106. As mentioned previously, in alternative embodiments, device 100 may include a guidewire lumen rather than guidewire coupler 106 and may thus be passed over a guidewire into the spine rather than being pulled behind a guidewire. In use, expandable portion 104 may be advanced partway into an intervertebral foramen, and then expander 105 may be advanced within expandable portion 104 using pusher 102 to expand expandable portion 104. Using radiography, depth markers and/or the like, a user may determine an approximate size of the intervertebral foramen based on how far expander 105 can be advanced along wire 101. As used in the present application, “approximating the size” of a foramen may mean approximating a cross-sectional area of the foramen, a volume of the foramen, a height or width of the foramen at one or more points, an amount of room a nerve root has within a foramen, and/or a cross-sectional area, volume, height or width of a portion of the foramen. In various embodiments, expandable portion 104 may be entirely radiopaque or include radiopaque markers and may be either closed on all sides or comprise two layers of material that expand away from one another.

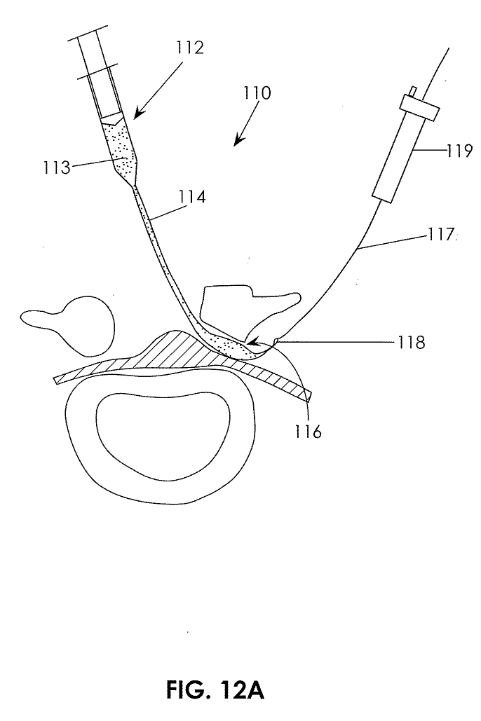

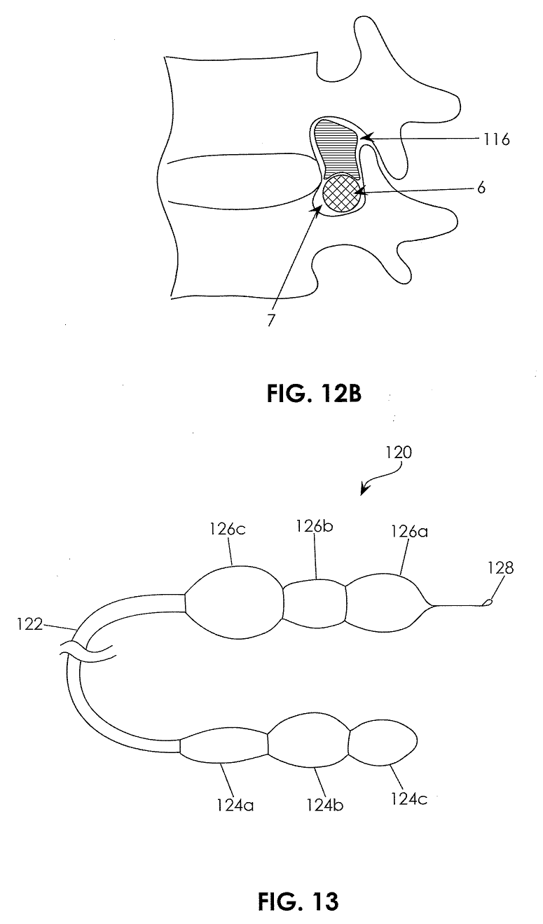

Measuring devices may also include inflatable or expandable regions. For example, FIGS. 12A and 12B show another embodiment of a device 110 for measuring an intervertebral foramen that includes an elongate flexible catheter 114 coupled with a fluid source 112 at its proximal end, having an inflatable balloon 116 at or near its distal end, and having a guidewire coupler tip 118. Device 110 may be coupled with a guidewire 117, which may in turn be coupled with a distal handle 119, and in some embodiment guidewire 117 and distal handle 119 may be provided with device 110 as a system. In use inflatable balloon 116 portion of catheter 114 may be advanced into an intervertebral foramen in its deflated state by pulling it behind guidewire 117. Fluid 113 may then be passed into inflatable balloon 116, such as by depressing syringe 112. The volume of an intervertebral foramen may be approximated, in one embodiment, by measuring the volume of fluid passed into inflatable balloon 116. Alternatively or additionally, volume of the foramen may be approximated by taking a radiographic image and using a radiopaque fluid 113, such as a contrast dye. Catheter 114 and balloon 116 may be made of any suitable material commonly known or hereafter discovered, such as any suitable polymer.

FIG. 12B shows a side view of a spine with the inflatable balloon 116 of device 110 shown in cross section in an intervertebral foramen 7, along with nerve root 6. As is visible in this figure, balloon 116 may sometimes conform to a shape of the foramen, thus providing a more accurate approximation of the size of the foramen than a rigid device.

With reference now to FIG. 13, in another embodiment, an intervertebral foramen measurement device 120 may include an elongate catheter 122 with a compartmentalized proximal balloon 124, a compartmentalized distal balloon 126, and a guidewire coupler tip 128. Distal balloon 126, for example, may have three compartments, to approximate the size of the vertebral central canal 126c, lateral recess 126b and foramen 126a. In one embodiment, each of those three compartments is replicated in proximal balloon 124, and fluid may be transferred under pressure from proximal balloon 124 to distal balloon 126. As the compartments of proximal balloon 124 empty, the compartments of distal balloon 126 fill until they can no longer fill because they have reached the size of the anatomical structures in which they reside. Thus, the size/volume of the proximal balloon 124 may provide a readout of the foramen by correlating with the size of the distal balloon component, without requiring the use of a visualization method such as fluoroscopy. The proximal balloons form a negative representation of distal balloon 126, thus reflecting the size and shape of the foramen, lateral recess and central canal. Compartments 124a, 124b, 124c, 126a, 126b, 126c may be separated, for example, by valves.

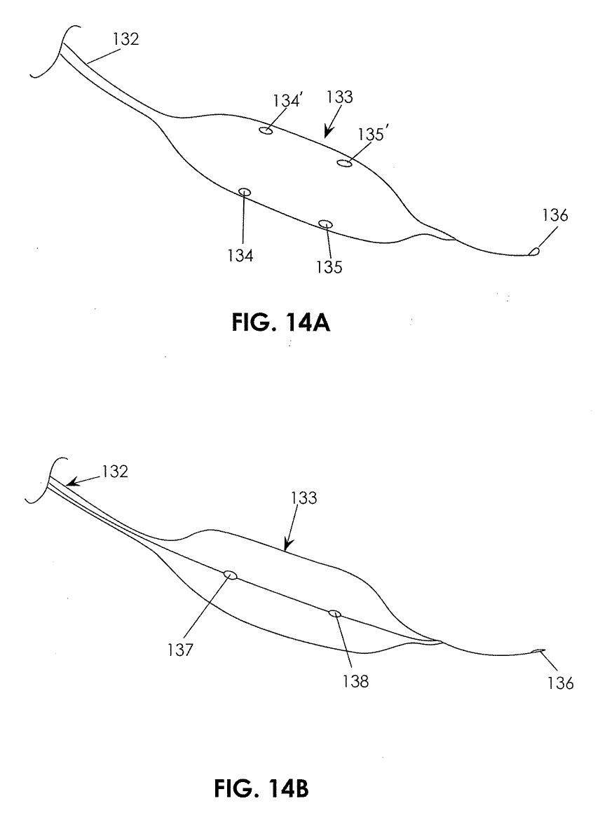

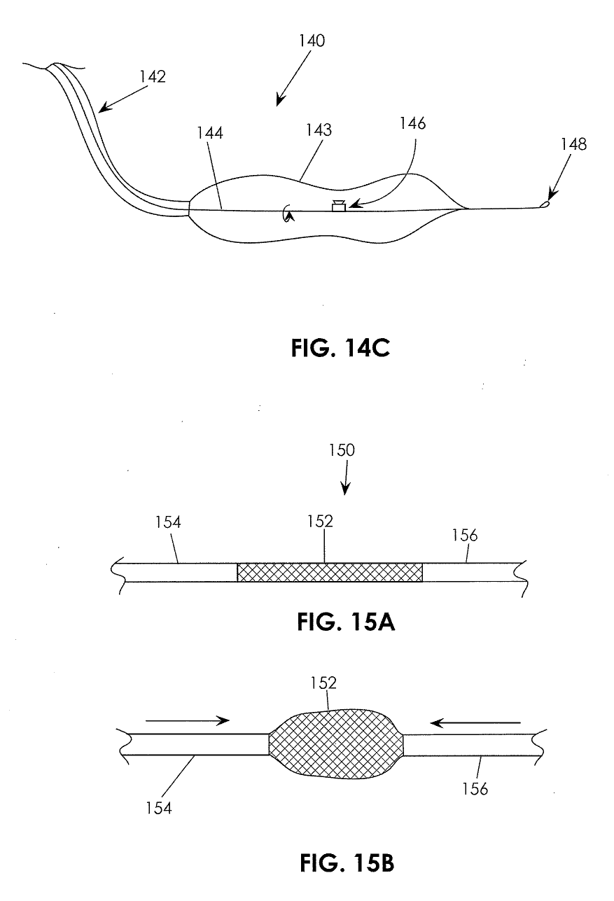

Another inflatable or expandable variation of a measuring device is illustrated in FIG. 14A. In this example, the intervertebral foramen measurement device 130 includes an elongate catheter 132, an inflatable balloon 133 disposed at or near a distal end of catheter 132, multiple electrodes 134, 134′ 135, 135′ coupled with balloon 133, and a guidewire coupler 136 disposed at or near a distal tip of device 130. Balloon 133 may be passed into an intervertebral foramen in a deflated state (e.g. by pulling it into position from the distal end of the guidewire). The measuring device may then be inflated to assume the shape of the foramen by passing a fluid, such as saline or any other biocompatible fluid, through catheter 132 into balloon 133. Once balloon 133 is inflated with fluid, current may be passed between various pairs of the electrodes (i.e., 134, 134′ and/or 135, 135′), and electrical properties measured to derive the distance between the electrodes. For example, the current passing between the electrodes may be analyzed to determine the rate of current passage between various electrodes to approximate the spacing of the electrodes, based on the known electrical properties of the fluid filling the (insulating) balloon. This may be used to derive distances between various electrode pairs over the balloon 133. Multiple electrodes may then be used to reconstruct a 3-dimensional image of balloon 133, thus approximating a shape of the foramen in which it has been inflated.

Similarly, FIG. 14B illustrates another variation of a measurement device in which current may be applied between two (or more) electrodes 137, 138 within an insulated balloon that has been inflated within the intervertebral foramen. Saline or other conductive material may be used to fill/inflate the balloon, and the volume of the balloon may be determined by the electrical properties. For example, an impedance measurement (taken at one or more frequencies) may be used to determine the volume within the balloon.

FIG. 14C shows another example of an inflatable device. In this variation, the device includes an inflatable region 143 located at the distal region of the device 140. The distal end of the device includes a coupler 148 for coupling to a guidewire. A flexible catheter including an inflation lumen connects the inside of the balloon to the proximal end of the device. A transducer 146 is positioned within the balloon. The transducer is configured to rotate (e.g., on a central axis or wire) to allow measurement of the distance to the inside of the balloon, from which the volume of the inflated balloon can be determined. In some variations the transducer is an optical transducer (e.g., camera), in other variations the transducer is an ultrasound transducer, or other modality transducer that may allow determination of the distance around the balloon.

With reference now to FIGS. 15A and 15B, in another alternative embodiment, an intervertebral foramen measurement device 150 may include elongate catheter proximal 154 and distal 156 portions with an expandable mesh 152 disposed between the two. In use, device 150 may be inserted into a patient and mesh 152 advanced into an intervertebral foramen in its unexpanded state, as shown in FIG. 15A. Proximal portion 154 and distal portion 156 may then be pushed toward one another to expand mesh 152 to assume the approximate shape of the foramen. Mesh 152 may be made of radiopaque material, and thus a radiographic image may be acquired (using intraoperative fluoroscopy, for example) to help approximate the size of the foramen. In some embodiments, multiple images may be taken, such as lateral, anterior-posterior and/or oblique views, to help approximate a shape of the foramen. In an alternative embodiment, it may be possible to pull on proximal portion 154 and distal portion 156 to expand mesh 152. Mesh 152 may comprise any suitable material, such as stainless steel, any other metal, polymer or the like. The distal end of this variation of a measurement device may be configured to couple with a guidewire so that it can be pulled through the intervertebral foramen and positioned therein. In some variations the guidewire may be coupled to the device so that it pressure can be applied distally (e.g., pushing against the distal end). In other variations the distal end of the device is configured to exit the subject so that it can be grasped and pressure can be applied thereto.

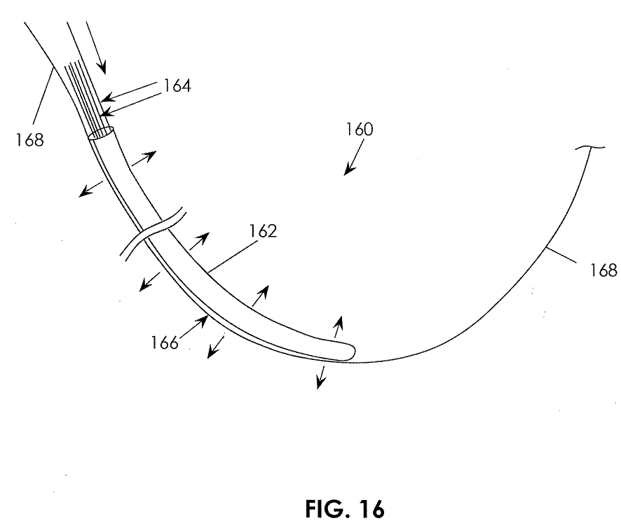

FIG. 16 illustrates another variation of an expandable measurement device. In this embodiment the device 160 for foramen measurement includes an expandable pouch 162 (or expandable catheter), multiple expansion members 164 (such as flexible wires, plates or the like), and a guidewire tube 166 (or guidewire lumen) coupled with pouch 162, so that device 160 may be advanced into a patient's body over a guidewire 168. A distal portion of pouch 162 may be advanced into an intervertebral foramen in an unexpanded state, with no expansion members 164 residing therein (or with few expansion members 164), and the expansion members 164 may be passed into pouch to cause it to expand. The size (e.g., inner diameter) of an intervertebral foramen may be approximated by the number of wires or other expansion members 164 that can be passed into pouch 162. Additionally or alternatively, in some embodiments, pouch 162 and/or expansion members 164 may be radiopaque and may therefore be imaged using radiographic imaging technique(s) to help approximate the size and/or shape of the foramen. Pouch 162 may be made of any expandable material, such as any of a number of different polymers. Expansion members 164 may be made of any suitable material, such as but not limited to stainless steel, Nitinol, other metals, polymers or the like.

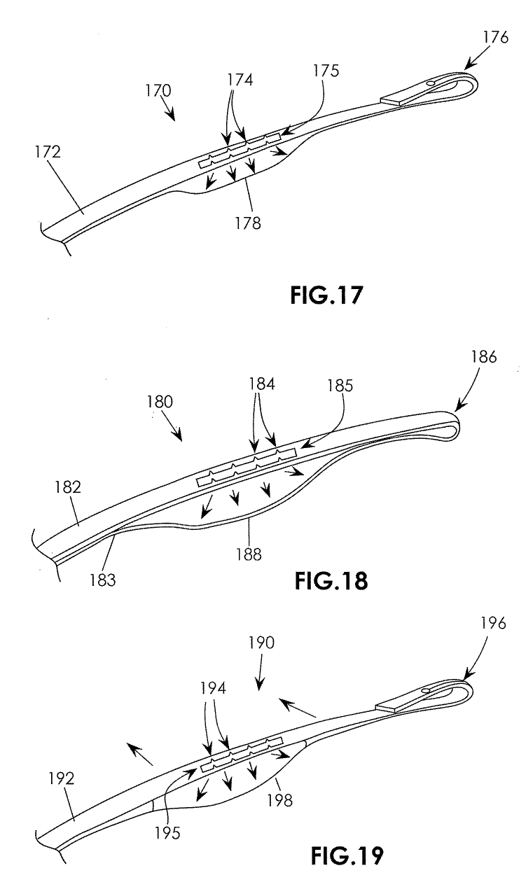

Any of the measurement devices described herein may be included as part of a system for decompressing nerves in the intervertebral foramen including a guidewire and a tissue removal device as described above. In some variations, the measurement device may be part of a tissue removal device. For example, FIG. 17 illustrates a tissue removal device 170 including a measurement feature. The tissue removal device is similar to that shown in FIG. 4. FIG. 17 shows a distal portion of such a tissue removal device 170, which may include a substrate 172 having upper and lower surfaces, multiple blades 174 formed from substrate 172, an aperture 175 (or “opening”) formed in substrate 172, a tissue collection pouch 178 disposed under the lower surface of substrate 172 in fluid communication with aperture 175, and a guidewire coupler 176. In this embodiment, tissue (such as ligamentum flavum, other soft tissue and/or bone) cut with blades 174 may pass through aperture 175 into pouch 178, thus expanding pouch 178. As pouch 178 expands, it may become increasingly difficult to reciprocate device 170 in the foramen, thus indicating to a user that a sufficient amount of tissue-has been removed and the procedure is complete. In some embodiments, all or a portion of pouch 178 may be radiopaque, so that as it expands a radiographic image may be taken of it to approximate a size and/or shape of the foramen.

Referring to FIG. 18, in an alternative embodiment, a tissue removal device 180 may include an upper layer 182 and a lower layer 183. An aperture 185 and multiple blades 184 may be formed in upper layer 182, such that aperture opens into a pouch 188 formed by lower layer 183. Device 180 may also include a guidewire coupler 186. Device 180 may work similarly to the previously described embodiment, with the size and/or shape of an intervertebral foramen being approximated by size and/or shape of pouch 188 as it fills, either by tactile feedback, radiographic images or both. In this or the previous embodiment, it may also be possible to remove device 180 (or 170) from the patient to directly visualize the size of pouch 188 (or 178) and/or to remove tissue from pouch 188 to assess its amount.

With reference now to FIG. 19, in another alternative embodiment, a tissue removal device 190 may include a substrate 192, multiple blades 194, an aperture 195, a guidewire coupler 196 and a side tissue collection pouch 198. In this embodiment, pouch 198 may be in fluid communication with aperture 195 but may be disposed asymmetrically on a side of lower surface of substrate 192, such that as pouch 198 fills with cut tissue, it pushes device 190 toward an opposite side of an intervertebral foramen. This may facilitate side-to-side/lateral movement of device 190 within an intervertebral foramen, which may help device 190 to remove a greater amount of tissue. The size and/or shape of the foramen may be assessed via pouch 198 as in the previously described embodiments.

As mentioned briefly above, any of the devices for measuring the intervertebral foramen may include neural stimulation. In particular, the device may include one or more tight bipole pairs configured to emit a localized stimulation field capable of activating a nearby nerve (e.g., the nerve root). Multiple bipole pairs may be associated with specific regions of the measurement device. Activation of the “tight” bipole field in a particular region will stimulate only a nearby (e.g., adjacent) nerve. A tight bipole field may be emitted when the bipole pairs are configured so that they are close to each other and are stimulated so that the current passed between the bipole pairs does not radiate substantially (i.e., less than a few millimeters from the surface of the measurement device). Thus, the nerve will be stimulated only when it is substantially close to the device (e.g., within contact or less than a 1 mm). Stimulation of the device may be detected by any appropriate methods, including (but not limited to) EMG measurement taken from the patient.

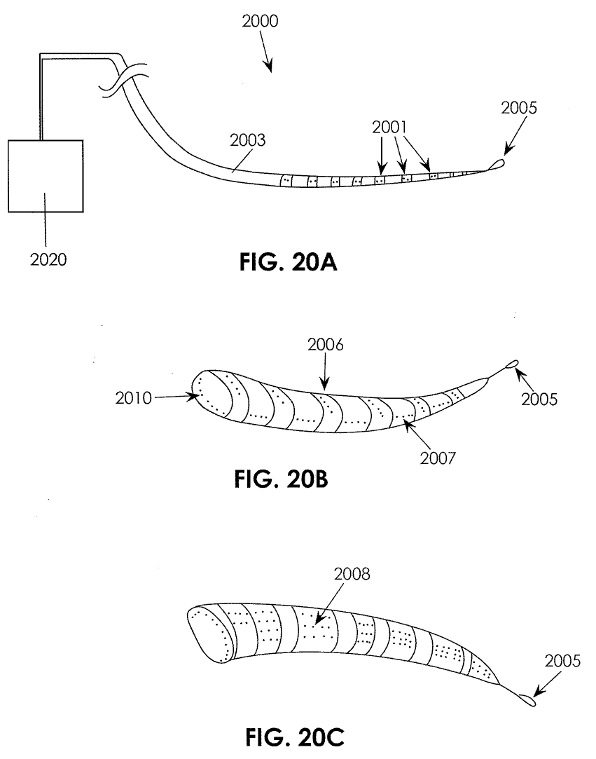

FIGS. 20A to 20C illustrate one variation of a measurement device 2000 including neural stimulation. In this example, the measurement device includes a tapered measurement probe. A handle may be located to the proximal end of the measurement device. The shaft portion 2003 extends distal to the handle; the distal region of the measurement device is tapered, and the very distal end of the device may include a coupling tip 2005 for coupling to a guidewire. The tapered region is typically divided up into different regions or zones 2001, Each zone may be a measurement region, having a specific diameter or range of diameters. For example, the taper in a specific region may be very slight. The zones may be marked with radio opaque bands or makers which allow the zones to be distinguished. Each zone may also include one or more bipolar pairs (e.g., tripolar pairs or a line of bipolar pairs) that may be activated by a stimulator 2020 to emit a bipole filed. Each of these zones or sections may be individually addressed (e.g., activated) by the stimulator or controller 2020.

FIG. 20B illustrates one variation of the distal region of a measurement device having neural stimulation. In this example, the distal end has a width that is less than the height (thickness), which may allow the device to more readily fit within the foramen. The distal end is divided up into different zones or regions that are longitudinally separated. In some variations, the zones or regions are also divided up into top/bottom/left side/right size sub-regions. Any of these zones/regions and sub-regions may be activated separately or at the same time. For example, all of the sub-regions of a particular longitudinal region may be activated at once. In some variations, each zone or sub-region includes a plurality of cathodes and anodes. Each of the anodes and/or cathodes may be separately connectable to a stimulator 2020 for controlled activation of a specific pair, or they may be grouped. For example, all of the anodes in one zone or sub-region may be connected to or part of the same anode. Similarly, all of the cathodes in one zone or sub-region may be connected to or part of the same cathode 2010. This may help reduce or simplify wiring of the device.

Because of the very small spacing between the bipole pairs (or tripoles), the device may precisely detect contact with a nerve. The bipole broadcast distance may be adjusted by varying the spacing of the bipoles, and/or the size of the bipoles. For example, the spacing between adjacent bipole pairs (anode and cathodes) may be less than 2 mm, less than 1 mm, less than 0.5 mm, etc. The surface area of each exposed anode/cathode may be less than 1 mm², less than 0.5 mm², etc. FIG. 20C illustrates the bottom side of the measurement probe shown in FIG. 20B. In FIGS. 20B and 20C, the bottom size may include more bipole pairs per zone. In some variations, it is expected that the nerve within the intervertebral foramen will be located on this side (e.g., anterior to the patents body) during the procedure.

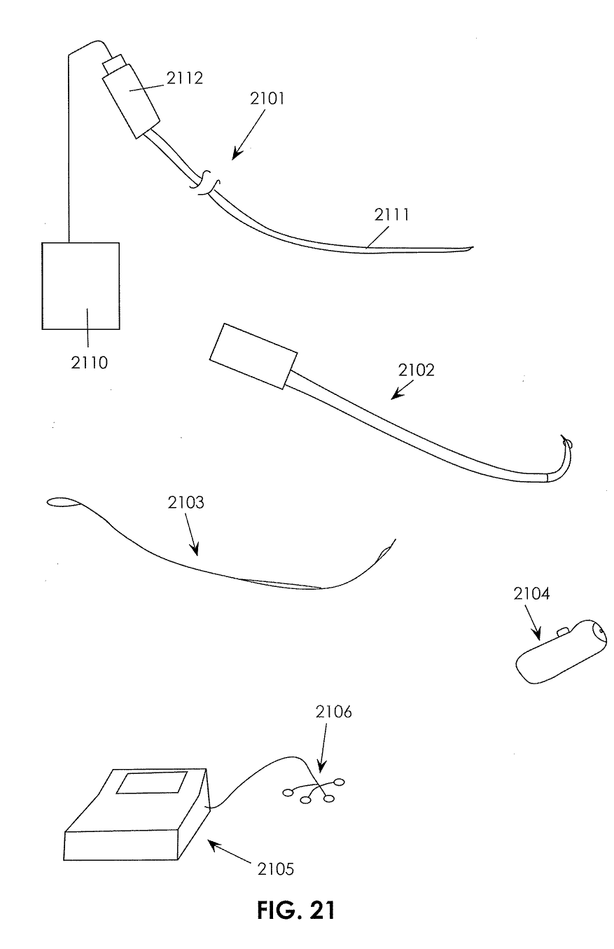

A measurement device including neural stimulation may be included as part of a system or kit, as mentioned above. FIG. 21 illustrates various components that may be included as part of a kit or system. For example, a kit or system may include a measurement probe with electrical bipoles 2101, and connections to a stimulator or controller 2110. The kit may also include a probe 2102 (e.g., a telescoping, curving probe) for positioning and delivering a guidewire 2103. The guidewire may include a sharp or pointed distal tip and a proximal end configured to be coupled to one or more devices. A handle 2104 may also be included for attaching to the distal end of the guidewire, as describe above with respect to FIG., 4. An EMG system (or subsystem) including an EMG reader 2105 and one or more probes or electrodes 2106, may also be included. The measurement probe may be a tapered probe, as illustrated in FIGS. 20A-21, or it may be configured as any of the measurement devices illustrated above including tight bipole pairs.

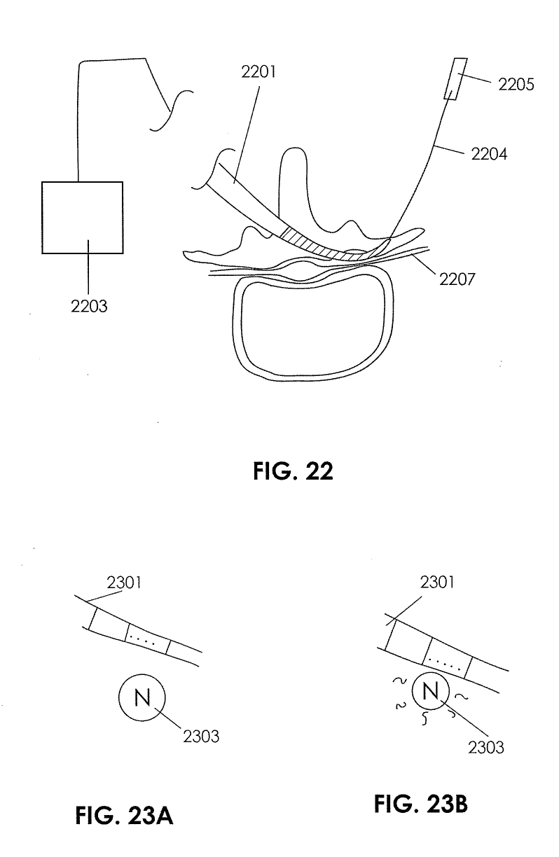

In operation, the measurement device may be inserted using the bimanual method described briefly above. For example, after introducing a guidewire from a first location outside of the patient, into and through the intervertebral foramen, and out of the patient at a second location, the proximal end of the guidewire may be coupled to the measurement probe. The guidewire may then be pulled (e.g., after attaching a handle) to draw the measurement probe through the intervertebral foramen. An exemplary illustration is provided in FIG. 22 for one variation of the measurement device. In this example, the measurement device is tapered, with marked regions each including neural stimulation that can be individually addressed. The distal end of the measurement device is drawn through the intervertebral foramen by pulling on the distal end (in this example, via the coupling to the guidewire).

In some variations, the measurement device may be pulled through the foramen until it cannot be advanced any further. The diameter of the foramen may then be estimated based on the marks on the measurement device. Neural stimulation can be used to determine the approximate diameter of the foramen adjacent to the nerve. Since decompression of the nerve (nerve root) is on goal of this procedure, it may be particularly important to know the diameter of this region. By selectively activating the bipole pairs nears in each zone, the zone nearest the nerve can be determined, and therefore the approximate dimension of the intervertebral foramen nearby (which must be at least as large as this zone or region).

In some variations, the measurement device may be advanced while stimulating the bipoles along the entire device. Since the bipole filed does not extend substantially from the surface of the device, neural stimulation of the nerve root will indicate when the device is approaching the nerve. This is illustrated in FIGS. 23A and 23B. For example, in FIG. 23A the bipole field originating from the measurement device 2301 does not activate the nerve 2303 because it is too far from the nerve to induce activation of the nerve. As the measuring device is advanced, and the taper of the device widens, the bipole filed approaches the nerve 2303 until it is stimulated, as indicated in FIG. 23B. By advancing the measurement device in this manner, (e.g., slowly) the size of the decompressed foramen may be estimated without damaging the nerve. Once activation has occurred, individual zone or regions of the measurement device may be stimulated to determine which zone or region is nearest to the nerve, and therefore what the approximate size of the foramen is.

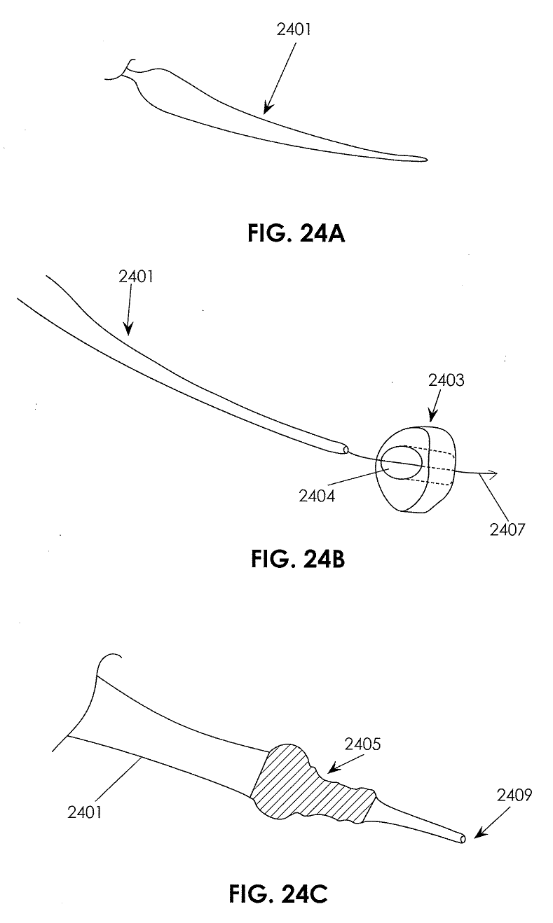

FIGS. 24A to 24C illustrate another variation of a measurement device 2401, including a shapeable or formable region at the distal portion of the device. In this example, the distal end is tapered. This sound region may be made of any appropriate, formable material. For example, the material may be a polymer. In some variations, the sound is made of a clay-like material (either synthetic or non-synthetic). For example, the sound may be made of a material that is moldable such as silicone plastic (putty of silicone and boric acid), or the like. Other exemplary materials may include PET, PE, PP, Urethone, FP, PTFE, Nylon, and co-polymers of any of these.

In some variations the measurement device includes a moldable inner core that is surrounded by a liner or outer film. This outer film or liner may be lubricious, and may eliminate direct contact between the moldable material and the patient's tissue.

FIGS. 24B and 24C illustrate one method of operation of a measurement device including a moldable or formable sound. As described above, the device may be used with a guidewire. For example, the distal end of the measurement device may include a coupler for coupling to the proximal end of a guidewire, so that the measurement device can be pulled through the foramen. In some variations the measurement device includes a guidewire lumen so that the device can be slid over the guidewire. In FIG. 24B, the measurement device 2401 is pulled through the intervertebral foramen 2403 by the guidewire 2407. The tapered end passes through the foramen, until the device is snuggly fitted into the foramen. This snug fitting may be determined by some minimum amount of force applied to draw it through the device. For example, the device may be limited to less than a few pounds of applied force (e.g., less than 10 lb of tension, less than 5 lbs of tension, less than 1 lb of tension, etc.). The measurement device may then be withdrawn by pulling on the proximal end of the measurement device (withdrawing the guidewire back through the foramen). FIG. 24C illustrates one example of a moldable sound region of a measuring device that has been placed into an intervertebral foramen until it has conformed to the shape of the foramen.

In FIG. 24C, a portion of the tapered formable distal end has taken on the shape of the intervertebral foramen 2405. The device will include a bulge near the proximal end where the material was prevented from entering the constricted foramen, and the region distal to this will have the maximum diameter shape of the narrowed region. A plateau region may be present, indicating the diameter of the foramen opening. This molded shape may then be measured to determine the dimensions, or compared with earlier/later (e.g., post-decompression or pre-decompression) sounds. In some variations the molded shape may be made permanent so that it can be later compared.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Furthermore, although many of the embodiments and variations described are directed to measuring the intervertebral foramina, these devices may be used or adapted for use in many other body openings, including other foramina, including general neural foramen.

Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

What is claimed is:

1. A method of measuring the size of a compliant region adjacent to a patient's nerve root, the method comprising:
advancing a guidewire from a first position outside of the patient's body, through an intervertebral foramen, and out of the patient's body at a second position;
advancing a measurement device adjacent to a portion of the nerve root, wherein the measurement device is coupled to the guidewire; and
estimating a size of the compliant region adjacent to the nerve root, based on the advancement of the measurement device.

2. The method of claim 1, wherein advancing the measurement device comprises pulling it into the intervertebral foramen and/or the lateral recess behind the guidewire, wherein the measurement device is coupled to the proximal end of the guidewire.

3. The method of claim 1, wherein multiple measurement devices are provided, each of a different diameter, and wherein estimating the size of the compliant region adjacent to the nerve root comprises determining a largest of the devices that can pass adjacent to the nerve root.

4. The method of claim 1, further comprising expanding an expandable region of the measurement device.

5. The method of claim 4, further comprising
passing fluid into the expandable region of the measurement device to expand the region; and
estimating the size based on an amount of fluid that can be passed into the expandable portion.

6. The method of claim 1, wherein estimating the size comprises estimating a cross sectional area of the intervertebral foramen.

7. The method of claim 1, wherein estimating the size comprises estimating a volume of the intervertebral foramen.

8. The method of claim 1, further comprising applying neural stimulation from the measurement device and monitoring for EMG signals.

9. The method of claim 1, further comprising molding a moldable region of the measurement device within the compliant region adjacent to the nerve root and withdrawing the molded region.

10. The method of claim 1, wherein guidewire is percutaneously advanced.

11. A method of measuring the size of a compliant region adjacent to a patient's nerve root as part of a spinal decompression procedure, the method comprising:
advancing a guidewire from a first position outside of the patient's body, through an intervertebral foramen, and out of the patient's body at a second position;
pulling the measurement device at least partially into the intervertebral foramen,
wherein the measurement device is coupled to the proximal portion of the guidewire;
expanding a portion of the measurement device; and
estimating a size of the compliant region adjacent to the nerve root, based on the expansion of the measurement device.

12. The method of claim 11, wherein the step of expanding a portion of the measurement device comprises expanding a portion of the measurement device within the intervertebral foramen.

13. The method of claim 11, wherein expanding the portion of the measurement device comprises passing a fluid into the portion.

14. The method of claim 13, wherein the fluid is passed into an expandable balloon of the measurement device.

15. The method of claim 13, wherein the fluid is passed into the portion until it reaches a predetermined pressure.

16. The method of claim 13, wherein the fluid is radiopaque, the method further comprising taking a radiographic image of the expanded portion using a radiographic device.

17. The method of claim 11, further comprising activating a transducer to estimate the size of the expanded portion.

18. The method of claim 11, wherein expanding the portion of the measurement device comprises passing an expansion member into an expandable portion of the device.

19. A measurement device for measuring the size of a compliant region adjacent to a patient's nerve root as part of a spinal decompression procedure, the device comprising:
a proximal end configured to be gripped;
a guidewire coupling region at the distal end, the guidewire coupling region configured to mate with the proximal end of a guidewire; and
a sound region near the distal end,
wherein the sound region is configured to be flexibly pulled at least partially through the intervertebral foramen and provide indication of the dimension of the intervertebral foramen.

20. The device of claim 19, wherein the sound region of the measurement device comprises a plurality of calibrated sounds of increasing dimension extending proximally from the distal region.

21. The device of claim 19, wherein the sound region of the measurement device comprises a calibrated tapered region.

22. The device of claim 19, wherein the sound region comprises a plurality of bipolar pairs configured to produce a bipole filed sufficient to activate an adjacent nerve.

23. The device of claim 19, wherein the sound region comprises an expandable region configured to be expanded within the intervertebral foramen.

24. The device of claim 23, wherein the expandable region is an inflatable balloon.

25. The device of claim 23, wherein the measurement device further comprises an expansion member configured to be advanced distally and expand the expandable region.

26. The device of claim 19, wherein the measurement device comprises a moldable region.

27. A system for measuring the size of a compliant region adjacent to a patient's nerve root as part of a spinal decompression procedure, the system comprising:
a guidewire having a distal end and a proximal end, and configured to pass from a first position outside of a patient's body, through an intervertebral foramen, and out of the patient's body at a second position; and
a measurement device including a sound region near the distal end, and a guidewire coupling region at the distal end, the guidewire coupling region configured to mate with the proximal end of the guidewire; wherein the sound region is configured to be flexibly advanced at least partially through the intervertebral foramen and provide indication of the dimension of the intervertebral foramen.

28. The system of claim 27, wherein the sound region of the measurement device comprises a plurality of calibrated sounds of increasing dimension extending proximally from the distal region.

29. The system of claim 27, wherein the sound region comprises a plurality of bipolar pairs configured to produce a bipole filed sufficient to activate an adjacent nerve.

30. The system of claim 27, wherein the sound region comprises an expandable region configured to be expanded within the intervertebral foramen.

31. The system of claim 30, wherein the expandable region is an inflatable balloon.

32. The system of claim 30, wherein the measurement device further comprises an expansion member configured to be advanced distally and expand the expandable region.

33. The system of claim 27, wherein the measurement device comprises a moldable region.

34. The system of claim 27, wherein the guidewire comprises a shaped proximal end for coupling with the first and second flexible wires.

35. A device for measuring an intervertebral foramen as part of a spinal decompression procedure, the device comprising:
a flexible wire passable through an intervertebral foramen and including a distal tip coupler for coupling with a guidewire; and
a distal tapered sound region fixedly coupled with the flexible wire for passing into the intervertebral foramen; wherein the tapered sound comprises a moldable material configured to hold the shape of at least a portion of the intervertebral foramen when withdrawn from the intervertebral foramen.

36. A device for measuring an intervertebral foramen as part of a spinal decompression procedure, the device comprising:
a flexible catheter passable into an intervertebral foramen and having proximal and distal ends;
an inflatable balloon disposed along the catheter at or near its distal end; and
a coupler disposed along the catheter at or near its distal end for coupling the catheter with a guidewire.

37. The device of claim 36, further comprising a transducer suspended on a wire passing through the inflatable balloon for measuring the inner dimensions of the balloon.

38. A device as in claim 36, further comprising a second balloon coupled with the catheter at or near its proximal end, wherein the second balloon forms a negative image of the inflatable balloon when the latter is inflated in the intervertebral foramen.

39. A device for percutaneously measuring an intervertebral foramen as part of a spinal decompression procedure, the device comprising:
a flexible catheter configured to pass through an intervertebral foramen, the catheter having proximal and distal portions, and an expansion region;
a plurality of long, flexible expansion members configured to pass into the expansion region, wherein the expansion region is configured to expand as the expansion members are passed therein; and
a guidewire coupling region configured to couple the catheter with a guidewire that can advance the catheter into the foramen.

40. A device as in claim 39, wherein the guidewire coupling region comprises a guidewire coupler at or near the distal end of the catheter for allowing the catheter to be pulled into the foramen behind the guidewire.

41. A device as in claim 39, wherein the guidewire coupling region comprises a guidewire lumen for allowing the catheter to be passed into the foramen over a guidewire.

42. A method of measuring the size of a compliant region adjacent to a patient's nerve root, the method comprising:
advancing a guidewire from a first position outside of the patient's body, through an intervertebral foramen, and out of the patient's body at a second position;
applying an electrical current between a pair of tight bipolar electrodes on a measurement device;
advancing the measurement device until the patient's nerve root is stimulated by the applied electrical current, wherein the measurement device is coupled to the guidewire; and
estimating a size of the region adjacent to the nerve root, based on the advancement of the measurement device.
